(19) United States
(12) Patent Application Publication
(10) Pub. No.: US 2025/0161122 A1
(43) Pub. Date: May 22, 2025

(54) Nasal Swab Apparatus for Applying a Cleaning Solution

(71) Applicant: John W. Polley, Maple City, MI (US)

(72) Inventor: John W. Polley, Maple City, MI (US)

(21) Appl. No.: 18/838,631

(22) PCT Filed: Feb. 20, 2023
(86) PCT No.: PCT/US23/62895

§ 371 Date: Aug. 15, 2024

Related U.S. Application Data
(60) Provisional application No. 63/268,315, filed on Feb. 22, 2022.

(51) Int. Cl. A61F 13/38; A61B 17/24

(52) U.S. Cl. CPC A61F 13/38; A61B 17/24; A61B 2017/246

(57) ABSTRACT

A nasal swab apparatus for cleaning, washing, decolonizing, prepping or sterilizing of human nose nasal vestibules, the apparatus comprising: a body extending along a longitudinal axis; at least one prong extending parallel to or along the longitudinal axis of the body, the at least one prong having an attachment end connected to the body and an opposite free end; a swab attached to the free end of the at least one prong; a nasal guide being a nasal columellar guide or a nasal alar guide, the nasal guide being connected to the body or to the at least one prong; and wherein the body is configured to be engaged by a human hand to position the swab into a selected one of the nasal vestibules of a human nose such that the nasal guide contacts an exterior region of the nose to limit an insertion depth of the swab.

This application claims the benefit of U.S. provisional patent application Ser. No. 63/268,315 filed Feb. 22, 2022.

### FIELD OF THE INVENTION

This invention relates to nasal swabs. More particularly, the invention is directed to single and multiple prong nasal swabs with a columellar (Nasal, Alar, Nasal Rim, Nasal Opening, Nasal Vestibular, Vestibular Rim) guard and a nasal cleaning solution and a method for the cleaning, washing, prepping, decolonizing, and/or sterilizing of the human nose and nasal vestibules.

### BACKGROUND OF THE INVENTION

This section provides background information related to the present disclosure which is not necessarily prior art.

The ongoing pandemic of coronavirus disease (Covid-19) caused by the SARS-CoV-2 virus and it's multiple variants have changed our world culture, perhaps forever. Efforts to avoid these highly contagious viruses have included travel restrictions, lockdowns, social distancing, masking, vaccines, vitamin regimens, personal protective equipment (PPE), and hygiene techniques. Hygiene is of paramount importance, and primary emphasis has been on hand washing and the avoidance of hand to face contact. A clear understanding of the pathophysiology of the infectious process, for both viral and bacterial infections, is essential in formulating appropriate hygiene methodologies.

Numerous studies have now clearly demonstrated that the major pathway in which the SARS-COV-2 virus (Covid-19), influenza virus, SARS virus, and MERS virus is initially transmitted to humans is through the upper respiratory tract by way of the nasal cavity (nose) (see Publications 1-9 identified below). Like most airborne viral diseases, the nose and upper airway are the first sentry point of infection (Publications 1, 2, 5-9). Nasal shedding of live viruses can be extreme in the early course of viral infections, precedes lower respiratory tract viral shedding, and can continue even after full symptomatic recovery (Publications 2, 5-11). Much higher viral loads are found in nasal swabs when compared to throat swabs (Publication 12). That is why Covid-19 testing is performed with nasal swab tests. This corresponds to the fact that 90%+ of inspiration is through the nose. Due to this pathogenesis rhinology and otolaryngology societies across the world have labeled nasal surgical procedures as high risk procedures (Publications 12, 13). It has been proven that the SARS-COV-2 virus and influenza virus infect the nasal cavity first, and its ability to infect and replicate progressively decreases as the virus proceeds further down the respiratory tract (Publications 2, 5-8). The virus enters the nose through two mechanisms. One is from direct contact with the emitting subject through airborne droplets (larger particles from contact within 6 feet) or aerosols (5um, higher distances than 6 feet) (Publications 5,13,14). The other is through hand to face contact with a hand contaminated with the virus droplets (Publications 2-5,15). SARS-COV-2 virus can also survive up to 72 hours on inanimate surfaces (Publications17,18). Once introduced to the nasal cavity viruses colonize in the anterior nose or nasal vestibule. The virus attaches to host nasal epithelial cells, replicates, and when reaching a critical mass invades further into the upper respiratory tract. Nasal vestibular epithelium and posterior nasal mucosal cells are abundant in angiotensin-converting enzyme 2 (Ace2) and transmembrane protease serine 2 (TMPRSS2), host proteins essential for the attachment to and infection of host target cells by SARS-COV-2, influenza and other viruses (Publications 2-5, 20, 21). The importance of the anterior nose or nasal vestibule in hosting the SARS-Co-V-2 virus is illustrated by the change in nasal swab techniques for testing for Covid-19.

Initially in the pandemic, the nasal swab testing technique included deep penetration of the swab into the nasal cavity, far beyond the posterior border of the nasal vestibule. Now the technique includes swabbing only of the nasal vestibule, not posterior to the anterior portion of the inferior turbinate (Publication 39). Pulmonary infection is believed to occur through micro-aspiration once the virus has reached sufficient numbers (Publications 1, 3-5). Penetration of the virus is variable amongst individuals. The incubation period in the nasal vestibules from initial nasal contagion until infection is variable, between 3-14 days (Publications 3, 20, 21). This pattern of initial viral nasal contamination, nasal incubation in the nasal vestibules, and consistent shedding, is similar to influenza and many other viral entities (Publication 12).

Viral shedding from the nasal cavity and upper respiratory tract is the primary mode of infection transmission (Publications 1-12). Early therapeutic elimination of viruses in the nasal cavity during the incubation period, prior to overt infection, is the objective. Early therapeutic elimination of colonizing viruses in the nasal cavity can significantly reduce the risk of person-to-person transmission as well as propagation of the infection to the lower respiratory tract and other organs.

This nasal pathophysiologic process of colonization, incubation, infection and transmission is true not only for viral infections but for bacterial infections as well. Nearly half the human population is a nasal carrier of staphylococcus aureus and numerous other gram positive and gram negative bacteria (Publications 41, 42). These bacteria are responsible for the majority of hospital and long term care facility acquired infections. They are also primarily responsible for post and peri-operative infections. Routine nasal vestibular hygiene has been demonstrated to reduce the incidence of these nosocomial infections (Publications 43, 44, 45).

With this understanding of the pathophysiology of the infectious process of viruses and bacteria we can develop a scientific approach to prophylactic hygiene. Nasal disinfection should be the key component of prophylactic hygiene following exposure to potential sources of airborne viruses and bacteria (crowds, airplanes, close public contact, known carriers, etc.). Few studies have examined this and these primarily reviewed mouth rinsing, nasal sprays, and nasal lavage techniques (Publications 36-38, 40). The medical and nonprofessional literature lacks studies on how to prep or sterilize the nose. The reason is that published authors in this field have zero experience on how to prep/sterilize/clean the nasal vestibules. What is needed is a simple, effective, well documented true surgically proven nasal cleaning and disinfecting technique with the appropriate utensils that can eradicate harmful bacteria, viruses, and allergic danders in the nasal vestibule during the early colonization phase, prior to overt infection.

Bibliography of publications on pathophysiology and disinfectants:
1. Zou L, et al. SARS-COV-2 viral load in upper respiratory specimens of infected patients. N Engl J Med 382(12), 1177-1179 2020
2. Gengler I, et al: Sinonasal pathophysiology of SAS-COV-2 and COVID-19: A systematic review of the current evidence. Laryngoscope Investig Otolaryngol, Vol 5(3): pp 354-359, June 2020
3. Wu D, et al. The SARS-COV-2 outbreak: what we know. Int J Infect Dis. 2020. 10.1016/j.ijid.2020.03.004
4. Young BE, et al. Epidemiologic features and clinical course of patients infected with SARS-COV-2 in Singapore. JAMA. 2020.10.1001/jama.2020.3204
5. Gallo O. et al: The central role of the nasal microenvironment in the transmission, modulation, and clinical progression of SAS-COV-2 infection. Mucosal Immunology, Vol 14, pp 305-316, 2021
6. Andersen, KG, et al. The proximal origin of SARS-COV-2. Nat Med 26,450-452 2020
7. Zou L, et al. SARS-COV-2 viral load in upper respiratory specimens of infected patients. N Eng J Med, https://doi.org/10.1056/NEJMc2001737 2020
8. Sungnak W. et al. SARS-COV-2 entry factors are highly expressed in nasal epithelial cells together with innate immune genes. Nat Med 26, 681-687 2020
9. Hou Y, et al. Researchers map how the coronavirus infection travels through cells of nasal cavity and respiratory tract. Gillings School News, Epidemiology News Un N Carolina Gillings School Global Pub Health 2021
10. Rothe C, et al. Transmission of 2019-nCoV infection from an asymptomatic contact in Germany. N Eng J Med 2020;382:970-971
11. Teleman MD, et al. Factors associated with transmission of severe acute respiratory syndrome among health-care workers in Singapore. EpidemiolInfect. 2004;132:797-803
12. European Rhinologic Society. Information for Rhinologists on COVID-19. https://www.europeanrhinologicsociety.org/id=2143 Accessed March 30,2020
13. American Academy of Otolaryngology-Head and Neck Surgery Academy SupportsCMS, OffersSpecificNasalPolicy https://www.entnet.org/content/academy-supports-cms-offers-specific-nasal-policy. Accessed March 30,2020
14. Van Doremalen N, et al. Aerosol and surface stability of SARS-COV-19 as compared with SARS-COV-1. N Engl J Med. 382, 1564-1567 2020
15. Asadi S, et al. The coronavirus pandemic and aerosols: does COVID-19 transmit via expiratory particles? Aerosol Sci Technol https://doi.org/10.1080/02786826.2020.1749229 2020
16. Sakurai A, et al. Natural history of asymptomatic SARS-COV-2 infection. N Engl J Med https://doi.org/10.1056/NEJMc2013020 2020
17. Stathis, C, et al: Review of the use of nasal and oral antiseptics during a global pandemic. Future Microbiol, 16(2): pp 119-130, January 2021
18. Kampf G, et al. Persistence of coronaviruses on inanimate surfaces and their inactivation with biocidal agents. J Hosp Infect 104 (3), 246-251 2020
19. Lauer SA, et al. The batiincuon period of coronavirus disease 2019 from publicly reported confirmed cases: astimation and applidation. NN INTERN MED HTTPS://DOI.org/10.7326/M20-0504 2020
20. ZIEGLER CG, ET AL SARS-COV-2 RECEPTOR E2 is an interferon-stimulated gene in human airway epithelial cells and is detected in specific cell subsets across tissues. Cell 2020
21. Chemical disinfectants: Guidelines for disinfection and sterilization in healthcare facilities. CDC 2018
22. Cegolon, L. et al: Nasal disinfection for the prevention and control of COVID-19: A scoping review on potential chemo-preventive agents. Int J Hyg Environ Health, September 2020; 230; 113605
23. Capetti, AF, et al: Short term inhibition of SAS-COV-2 by hydrogen peroxide in persistent nasopharyngeal carriers. J. Medical Virology; Vol 93 (3) U.S. Plant Pat. No. 1,766-1769 Sept 2020
24. Do disinfectants kill newer variants of coronavirus? EPA.gov 2021
25. Choon Goh, et al: Dermatologic reactions to disinfectant use during the COVID-19 pandemic. Clin Dermatol; 39(2); pp314-322 March 2021
26. Darouiche RO, et al. Clorhexidina-Alcohol versus Povidone-lodine for surgical antisepsis N Engl J Med 2010, 362:18-26
27. Komine A, et al. Virucidal activity of oral care products against SARS-COV-2 in vitro. J Oral Maxfac Surg, Med, and Path (33) #4, 475-477 July 2021
28. Huang YH, et al. Use of chlorhexidine to eradicate oropharyngeal SARS-COV-2 in COVID-19 patients. J Med Virol 93(7) 4370-4373 July 2021
29. Bernstein D, et al. In vitro virucidal effectiveness of a 0.12%-chlorhexidine gluconate mouthrinse. J Dent Res 69(3): 874-876 March 1990
30. Moosavi MS, et al. Antiviral mouthwashes: possible benefit for COVID-19 with evidence-based approach. J Oral Microbiol 12(1) 1794363 2020
31. Gottsauner MJ, et al. A prospective clinical pilot study on the effects of a hydrogen peroxide mouthrinse on the intraoral viral load of SARS-COV-2 Clin Oral Investig 24(10) 3707-3713 2020
32. Caruso AA, et al. Might hydrogen peroxide reduce the hospitalization rate and complications of SARS-COV-2 infection? Infect Control Hosp Epidemiol 2020:1-2. Online April22, 2020.doi: 10.1017/ice.2020.170
33. Stull JD, et al. Addition of 3% hydrogen peroxide to standard skin preparation reduces Cutibacteriuum acnes-positive culture rate in shoulder surgery: a prospective randomized controlled trial. J Shoulder and Elbow Surg. 29, 212-216 Urban V, et al. Hydrogen peroxide (H2O2): a review of its use in surgery. Wien Med Wochenschr 169:222-225 2019
34. Urban V, et al. Hydrogen peroxide (H2O2): a review of its use in surgery. Wien Med Wochenschr 169:222-225 201
35. Goh CF, et al. Dermatologic reactions to disinfectant use during the COVID-19 pandemic. Clin Dermatol 39(2): 314-322 March-April 2021
36. Tucker SP, et al. In vitro inactivation of SARS-COV-2 with 0.5% povidone iodine nasal spray (Nasodine) at clinically relevant concentrations and timeframes using tissue culture and PCR based assays. BioRxiv doi: http://doi.org/10.1101/2021.01.31.426979
37. Linley E, et al. Use of hydrogen peroxide as a biocide: new consideration of its mechanisms of biocidal action. J Antimicrob Chemother 67:1589-1596 April 2012
38. Farrell NF, et al. Benefits and safety of nasal saline irrigations in a pandemic-washing COVID-19 away. JAMA Orolaryngol Head Neck Surg. 146 (9): 787-788 2020
39. Li M, et al. Comparing SARS-COV-2 testing in anterior nasal vestibular swabs vs oropharyngeal swabs. Cell Infect Microbiol. https://doi.org/10.3389/cfimb.2021.653794
40. Kramer G, et al. Mouthwashes, oral rinses may inactivate human coronaviruses. https://georgekramermd.us7.list-manage.com/track/click?u=574ff841f4e9844cebc
41. Bode LG, et al. Preventing surgical-site infections in nasal carriers of Staph. Aureus. N Engl J Med 2010:362:9-17.
42. Wertheim HF, et al. The role of nasal carriage in Staph. Aureus infections. Lancet Infect Dis 2005:5:751-62.
43. Steed LL, et al. Reduction of nasal Staph Aureus carriage in health care professionals by treatment with a nonantibiotic, alcohol-based nasal antiseptic. Amer J Inf Control 2014:42:841-6.
44. Huang SS, et al. Targeted vs universal decolonization to prevent ICU infection. N Engl J Med 2013:368:2255-65.
45. Septimus EJ. Nasal decolonization: What antimicrobials are most effective prior to surgery? Amer J Inf Control 2019:47:A53-A57

### BRIEF SUMMARY OF THE INVENTION

According an embodiment of the present invention, a nasal swab includes a columellar (Nasal, Alar, Nasal Rim, Nasal Opening, Nasal Vestibular, Vestibular Rim) guard or stop, and an antiseptic or disinfectant cleaning solution. Suitable solutions include:

Hydrogen Peroxide H2O2—Listed as a disinfectant by CDC. 1-3% FDA approved. Mouth rinse recommended by and used by American Dental Association (ADA). Is active against a wide range of microorganisms, including bacteria, yeasts, fungi, viruses, and spores. Viricidal for SARS-COV-2 virus. A 0.5% accelerated concentration demonstrated bactericidal and veridical activity in 60 seconds, and mycobactericidal and fungicidal activity in 5 minutes. A stabilized 7% concentration was viricidal in 5 minutes. Colorless and odorless. Is extremely stable under normal conditions when stored in dark containers. Decomposition or loss of potency in small containers is less than 2% per year at ambient temperatures.

Ethyl Alcohol—Listed as a disinfectant by CDC. FDA approved. Need 60%+ concentration to be effective for bactericidal effect, bactericidal activity drops sharply below 50% concentration. However, studies have shown concentrations of 30% or above inactivate SARS-COV-2 virus in 30 seconds. Cannot penetrate protein-rich materials so lack sporicidal action, therefore not recommended for surgical instrument sterilization. Prolonged exposure is harsh on industrial surfaces, especially rubber, plastic, etc. Volatile, evaporates rapidly, flammable, stored in cool environment. Active ingredient in Nozin Nasal Sanitizer. Recent studies have shown only a transient (8 hour) reduction of colonized nasal S. Aureus following three applications of Nozin Nasal Sanitizer (although they claim 90% bactericidal for 12 hours).

Isopropyl Alcohol—Listed as a disinfectant by CDC. FDA approved. Need 60%+ concentration to be effective for bactericidal effect, bactericidal activity drops sharply below 50% concentration. However, studies have shown concentrations of 30% or above inactivate SARS-COV-2 virus in 30 seconds. Cannot penetrate protein-rich materials so lack sporicidal action, therefore not recommended for surgical instrument sterilization. Prolonged exposure is harsh on industrial surfaces, especially rubber, plastic, etc. Volatile, evaporates rapidly, flammable, stored in cool environment. Strong smell.

Methyl Alcohol—Methyl alcohol (methanol) has the weakest bactericidal and viricidal action of the alcohols (vs. ethyl and isopropyl) so is seldom used in health care.

Phenethyl Alcohol—FDA approved. Weak bactericidal and viricidal action. Is highly volatile unless stabilized with additives. Used for its fragrant rose-smelling quality in foods, cosmetics, cigarettes, etc. Main ingredient in current commercial product Zicam Nasal All Clear.

Polyvinylpyrrolidone-Iodine (PVP-I)—Listed as a disinfectant by CDC. FDA approved. Povidone iodine (PVP-I) has been marketed as a disinfectant since 1955.PVP-I antiseptic solution 10%, skin cleanser 7.5%, gargle and mouth wash 1%, and throat spray 0.25% achieved 99.9% vericidal activity against SARS-COV-2 within 30 seconds of contact. Nasodine (0.5% PVP-I) is a commercially available nasal spray. Not recommended for pregnant women and children due to iodine release.

Chlorhexidine Gluconate—FDA approved. Has been used in medicine as a surgical prep solution since the 1950′s. Is widely used as a disinfectant in hand and skin sanitizers, cosmetics, as an additive to creams, toothpastes, deodorants and pharmaceutical products such as eye drops and mouthwashes. Is strongly bactericidal and virucidal. Has proven virucidal effects on enveloped viruses including SARS-COV-2 at concentrations at or above 0.12% (Most surgical skin prep solutions contain 2% Chlorhexidine). Frequently combined with Isopropyl alcohol to greatly augment its cidal activity.

Nasal sprays and nasal lavage are not adequate for fully cleaning the anterior nasal vestibules. Sprays do not scrub. Sprays can miss areas (inserting spray bottle too far into vestibule will miss completely the anterior portion of the vestibule). Sprays will not consistently penetrate to the basal layer of the epithelium. Sprays retain all materials in the vestibules, not removing anything. The only adequate method for cleaning the nose is through a true surgical prep approach. A precise, effective surgical nasal prep requires a double prep (Publication 26). This includes thorough cleaning followed by disinfecting or decolonizing.

A complete nasal surgical prep system, with instructions on use, is the goal of the nasal swab apparatus according to the invention. It offers the following advantages:
Professional (MD) surgical nasal prep cleaning
Easy to use, foolproof
Compact, lightweight, disposable Inexpensive, safe
Can be used anytime, anywhere without social concern
No need for sinks, water, private space to use
Assures proper placement of the device only in the nasal vestibule

A device according to the invention can consist of a handle, or finger cap (thimble), or finger sleeve, a prong, a swab, and or various nasal guides. The handle is for holding the device with human fingers or hands. Alternatively a finger cap (finger thimble), or a finger sleeve can be employed as the handle. The swab is for intranasal/intra nasal vestibular use or placement, the prong can connect the handle to the swab, and the nasal guides or stops are for guiding appropriate device placement using external nasal landmarks (columella, ala, nasal rim, nasal tip) for reference points. The guides or stops prevent the swab from being positioned too deep or too shallow in the nasal vestibule(s). Accurate anatomic placement of the swab(s) ensures thorough cleaning/scrubbing/prepping/disinfecting/decolonizing of the entire nasal vestibule(s). The device can include all of these components or any combination of these components.

The device can be a single, dual, or multipronged/multi swab/multi nasal guide device/tool/apparatus/mechanism as outlined above. The device can be constructed from any type of material such as paper or paper product, plastic of any type, metal, synthetic product or paper. The device can be made of biodegradable or non-biodegradable materials. The device has attached to or as part of it a swab, pad, wipe, gauze, wad, foam, sponge, cushion, mop, bolster, and/or covering. The attachment can be at any location on the device, either on an end of the device or anywhere along the body of the device.

The device swab can hold, absorb, carry, transport, transfer, pass on, spread, conduct, diffuse, be saturated by, soak-up, or be infused with any FDA approved antiseptic or disinfectant or combination thereof, with or without a moisturizing agent. The device can be formed in various configurations, shapes, designs, and arrangements as described below. The device can include one or more swabs, prongs, handles, and nasal guides or guards. The swab can be made of any type of absorbent material such as cotton, foam, sponge, synthetic fiber, gauze, etc. The device can be marketed as a final complete entity (in one piece), or as multiple individual components that are then assembled to create the final entity. For example the nasal swabs and prongs with or without a nasal guide may come separate from the handle. The two components are then attached (in any way possible) to create the final intact device. Each individual component (swab, prong, guide, handle) may be delivered on their own, or in any combinations, and then assembled into the final device.

The device components can be of various dimensions and shapes. The swab dimensions can be between 1×1×1 mm (1 cubic mm)-50×50×50 mm (5.0 cubic cm), or any dimension in between, or a volume of the swab anywhere between 1 and 50 cubic mm's. The swab configuration can be of any design, e.g. spherical, conical, oval, oblong, cuboid, pyramidal, etc. The handle, or finger cap dimensions can be between 1×1×1 mm-10×10×10 cm or any dimension in between. The handle configuration can be of any suitable design. The nasal guard or guides can be of dimensions 1×1×1 mm-50×50×50 mm or any dimension in between. The nasal guides between the prongs are the columellar guides; nasal guides extending out lateral to the prongs are the alar guide. The nasal prong can be of dimensions between 1×1×1 mm-10×10×10 cm or any dimension in between. The prong configuration can be of any suitable design. The prongs, handles, caps, and nasal guides can be formed in a die by a molding process.

An alternative configuration is the finger sleeve. With the sleeve the outer layer of the sleeve acts as the swab and holds or contains the antiseptic solution(s). Additionally antiseptic solution(s) can be added to the inside layer of the finger cap or finger sleeve to act as a disinfectant for the finger.

The device prongs, handles, and nasal guides can each be adjustable in dimension and configuration. The prongs, handles, and nasal guides can be adjustable either individually or in any combination.

The device can be packaged in, held in, delivered in, wrapped in, parceled in, a plastic or paper or metallic (or other synthetic based material) container with rip off or seal access. The package can contain antiseptic(s) that soak the swab(s) of the device in a various fashions. The package can contain one, two, or more of the devices.

The device can be used to perform a method for the cleaning, washing, prepping, decolonizing and/or sterilizing of the human nose regardless of the sex or age of the person. The method can be performed on the nasal vestibules. The method according to the invention can be performed using the inventive device having a single swab, or double, triple, quadruple, etc. swabs. The method can be used in medicine as a surgical prep/sterilization technique. The method can be used for commercial or public use enabling definitive nasal hygiene. The method can utilize alone or in any combination, FDA approved surgical prep antiseptics or disinfectants and/or moisturizing agents.

An aspect of the invention relates to a nasal swab apparatus for cleaning, washing, prepping, decolonizing and/or sterilizing of human nose nasal vestibules. The nasal swab apparatus includes: a body extending along a longitudinal axis; at least one prong extending parallel to or along the longitudinal axis of the body, the at least one prong having an attachment end connected to the body and an opposite free end; a swab attached to the free end of the at least one prong; a nasal guide being a nasal columellar guide or a nasal alar guide, the nasal guide being connected to the body or to the at least one prong; and wherein the body is configured to be engaged by a human hand to position the swab into a selected one of the nasal vestibules of a human nose such that the nasal guide contacts an exterior region of the nose to limit an insertion depth of the swab. The body can include a receptacle and the attachment end of the at least one prong is fixed in the receptacle or is removably retained in the receptacle.

The at least one prong can be a first prong extending parallel to the longitudinal axis of the body, and a second prong can extend parallel to the longitudinal axis of the body, the second prong having an attachment end connected to the body and a free end, the second prong being spaced a predetermined distance from the first prong, and including another swab attached to the free end of the second prong.

The nasal columellar guide either is formed as a portion of an attachment edge of the body between where the first and second prongs are connected to the body or extends between the first and second prongs and is spaced from the body. The nasal alar guide either extends from the body or extends from one of the first and second prongs.

The body and/or the prongs are constructed from a material selected from a paper, a paper product, a plastic, a metal and a synthetic product. The material can be biodegradable.

The swab is adapted to hold a quantity of an antiseptic or a disinfectant and to release the antiseptic or the disinfectant upon contact with a surface in the nasal vestibule of the human nose. The nasal swab apparatus can include a frangible ampule containing the antiseptic or the disinfectant, the frangible ampule being positioned in the prong or in the body, wherein when the frangible ampule is broken, the antiseptic or the disinfectant is delivered to the swab.

The nasal swab apparatus can include an adjustment mechanism positioned in the body and enabling selective adjustment of the body in a direction of the longitudinal axis. The nasal swab can include an adjustment mechanism positioned in the prong and enabling selective adjustment of the prong in a direction of the longitudinal axis of the body or in a direction transverse to the longitudinal axis of the body.

### BRIEF DESCRIPTION OF THE SEVERAL VIEWS OF THE DRAWING

The above as well as other advantages of the invention will become readily apparent to those skilled in the art from the following detailed description of a preferred embodiment when considered in the light of the accompanying drawings in which:

FIG. 1 is a front view of a nasal swab apparatus according to the invention;

FIG. 2 is a front view of alternate embodiments of the nasal swab apparatus shown in FIG. 1;

FIG. 3 shows various embodiments of the nasal swab apparatus of FIG. 1 in schematic form;

FIG. 4 is a schematic view of various embodiments of a single prong nasal swab apparatus according to the invention;

FIG. 5 is a schematic view of various embodiments of a four prong nasal swab apparatus according to the invention;

FIG. 6 is a schematic view of various embodiments of an adjustable nasal swab apparatus according to the invention;

FIG. 7 is a schematic view of various embodiments of a thimble style nasal swab apparatus according to the invention;

FIG. 8 is a schematic view of two embodiments of a single prong thimble style nasal swab apparatus according to the invention;

FIG. 9 is a schematic view of two embodiments of a nasal swab apparatus having an integral disinfectant dispenser; and

FIG. 10 is a schematic view of a nasal swab kit according to the invention.

### DETAILED DESCRIPTION OF THE INVENTION

The U.S. provisional patent application Ser. No. 63/268,315 filed Feb. 22, 2022 is incorporated herein by reference.

The above as well as other advantages of the present invention will become readily apparent to those skilled in the art from the following detailed description of a preferred embodiment when considered in the light of the accompanying drawings. The following detailed description and included drawings describe and illustrate various exemplary embodiments of the invention. The description and drawings serve to enable one skilled in the art to make and use the invention, and are not intended to limit the scope of the invention in any manner. In respect of the methods disclosed, the steps presented are exemplary in nature, and thus, the order of the steps is not necessary or critical.

More particularly, the invention is directed to single and multiple prong nasal swab devices with a columellar guard, a nasal cleaning solution, and a method for the cleaning, washing, prepping and/or sterilizing of the human nose nasal vestibules.

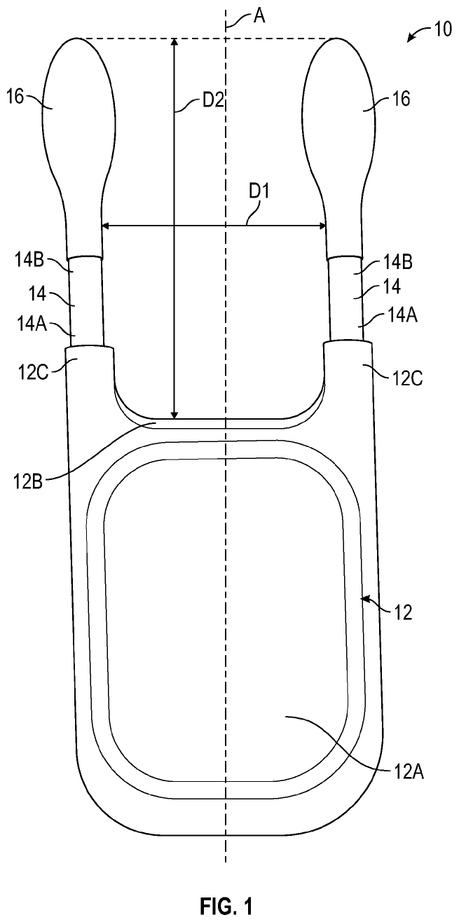

Shown in FIG. 1 is front side of a first embodiment of the nasal swab device 10 according to the invention. The rear side (not shown) can be identical. An elongated body 12 has a longitudinal axis A and is shaped as a handle or stem to be grasped between a thumb and a forefinger of a human hand (not shown). The body 12 can have central depressions 12A formed on opposite sides thereof as gripping aids. An attachment edge 12B of the body 12 has a pair of prongs or arms 14 extending therefrom generally parallel to the longitudinal axis A. The prongs 14 are spaced apart at a predetermined distance D1 to permit insertion into the nostrils of a human nose. Each of the prongs 14 has an attachment end 14A connected to the body 12 at the attachment edge 12B. The prongs 14 can be formed integral with the body 12. In the alternative, the prongs 14 can be separate parts with the attachment ends 14A either attached directly to the edge 12B or inserted into receptacles 12C extending from the edge 12B as shown in FIG. 1. When inserted in the receptacles 12C, the prongs 14 can be fixed or can be removable for replacement. Although the prongs 14 are shown as being of approximately the same length, the could be formed with different lengths.

Each of the prongs 14 has a free end 14B opposite the attachment end 14A. Each of the free ends 14B has an associated swab 16 attached thereto. Thus, the nasal swab device or apparatus 10 can be engaged by a human hand at the body 12 to position the swabs 16 simultaneously into separate ones of the nasal vestibules of a human nose. The portion of the attachment edge 12B that extends transversely between the attachment ends 14A functions as a nasal columellar guide, guard or stop. The nasal columellar guide limits an insertion depth D2 of the prongs 14 and swabs 16 into the nasal vestibules.

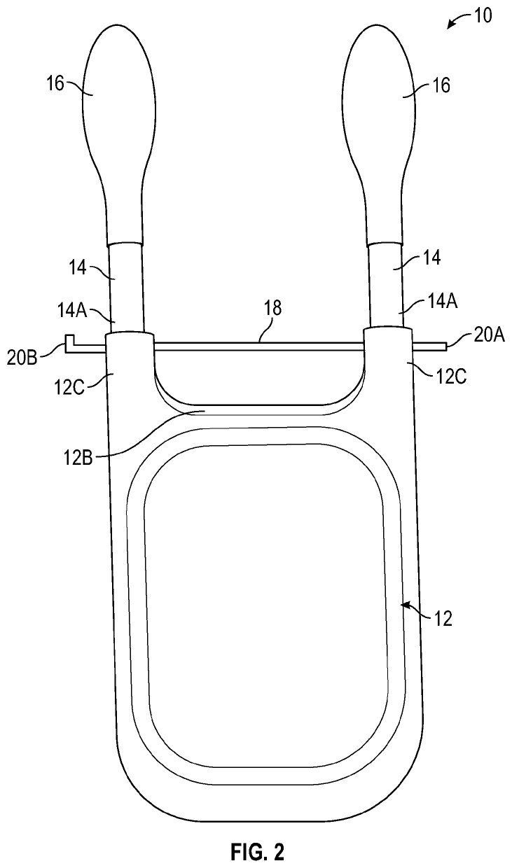

FIG. 2 shows two variations of the two prong nasal swab device 10 shown in FIG. 1. In a first variation, instead of the portion of the attachment edge 12B that extends transversely between the attachment ends 14A functioning as the nasal columellar guide, guard or stop, a separate nasal columellar guide 18 is spaced from the attachment edge 12B and extends between the receptacles 12C. In a second variation, a nasal alar guide, guard, stop is provided at each of the receptacles 12C. A nasal alar guide 20A extends from the right side receptacle 12C transverse to a longitudinal axis of the adjacent prong 14. A nasal alar guide 20B extends from the right side receptacle 12C transverse to a longitudinal axis of the adjacent prong 14. The guide 20B has an L-shape or could be curved. The nasal swab device 10 can be provided with any one or more of the guides 18, 20A and 20B.

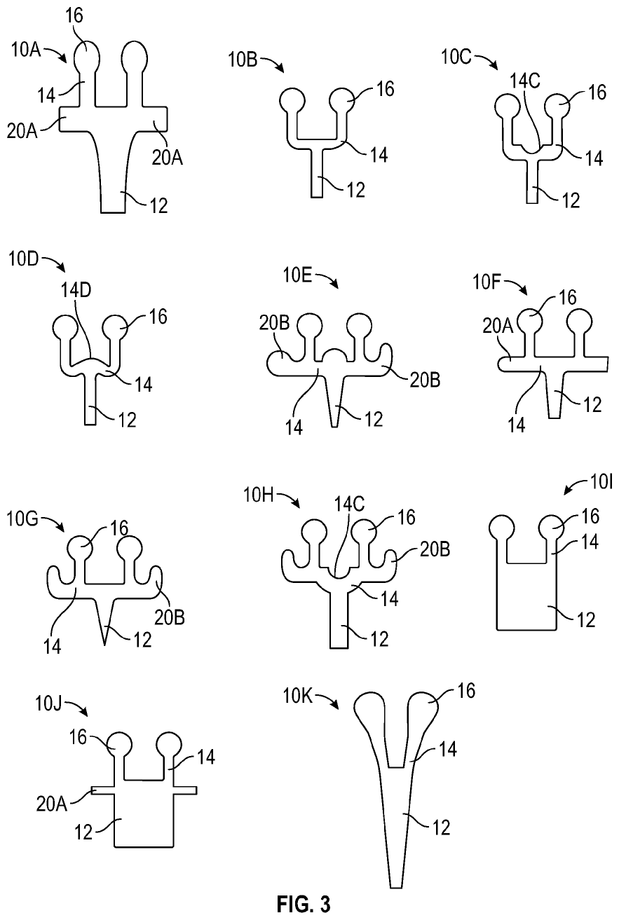

FIG. 3 shows various configurations of the nasal swab apparatus 10 in schematic form. For example, the apparatus 10A has the nasal alar guides 20A extending from the body 12. The apparatus 10B has a narrow, stem-like body 12 and L-shaped prongs 14. The apparatus 10C is similar to the apparatus 10B, but with a central recess 14C adjacent to the body 12. The apparatus 10D is similar to the apparatus 10B, but with a central protrusion 14D adjacent to the body 12. The apparatus 10E is similar to the apparatus 10D, but with two versions of the nasal alar guide 20B attached to the prongs 14. The apparatus 10F is similar to the apparatus 10B, but with the nasal alar guides 20A attached to the prongs 14. The apparatus 10G is similar to the apparatus 10E, but with the central protrusion 14D removed. The apparatus 10H is similar to the apparatus 10G, but with the central recess 14C added. The apparatus 101 is similar to the apparatus 10 shown in FIG. 1, but with the receptacles 12C removed. The apparatus 10J is similar to the apparatus 101, but with the guides 20A added. The apparatus 10K is similar to the apparatus 101, but with a narrower body 12 and with the prongs 14 angled outwardly. The configurations 10A through 10K are not exhaustive of the combinations of body, prongs, columellar guide and alar guides that will enable the nasal swab apparatus according to the invention to function.

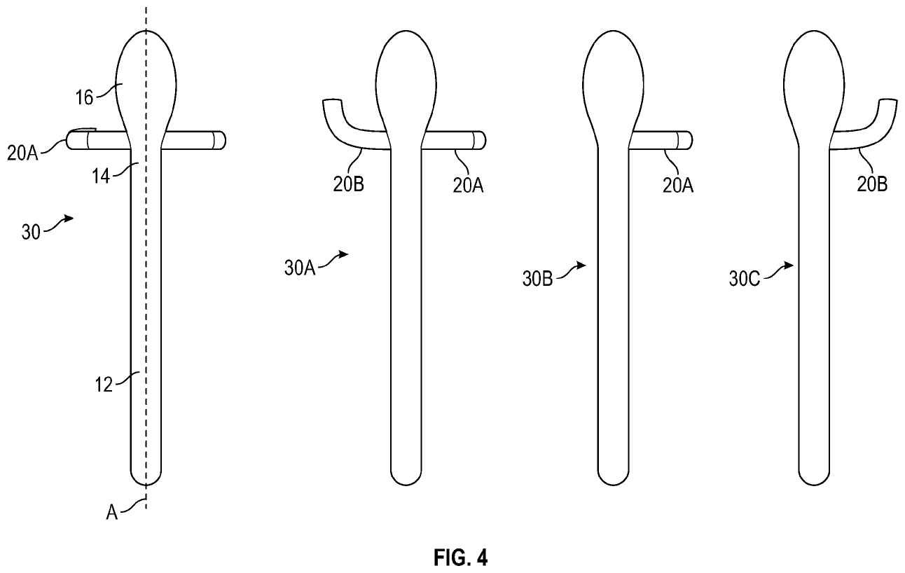

FIG. 4 is a schematic view of various configurations of a single prong nasal swab apparatus according to the invention. An apparatus 30 has the body 12 and the single prong 14 extending along the longitudinal axis A. A pair of nasal alar guides 20A are attached to the prong 14. An apparatus 30A is similar to the apparatus 30 with the nasal alar guide 20B replacing the left guide 20A. An apparatus 30B is similar to the apparatus 30 with the left guide 20A removed. An apparatus 30C is similar to the apparatus 30B with the guide 20A replaced by the guide 20B. Other configurations of the single prong nasal swab apparatus are possible.

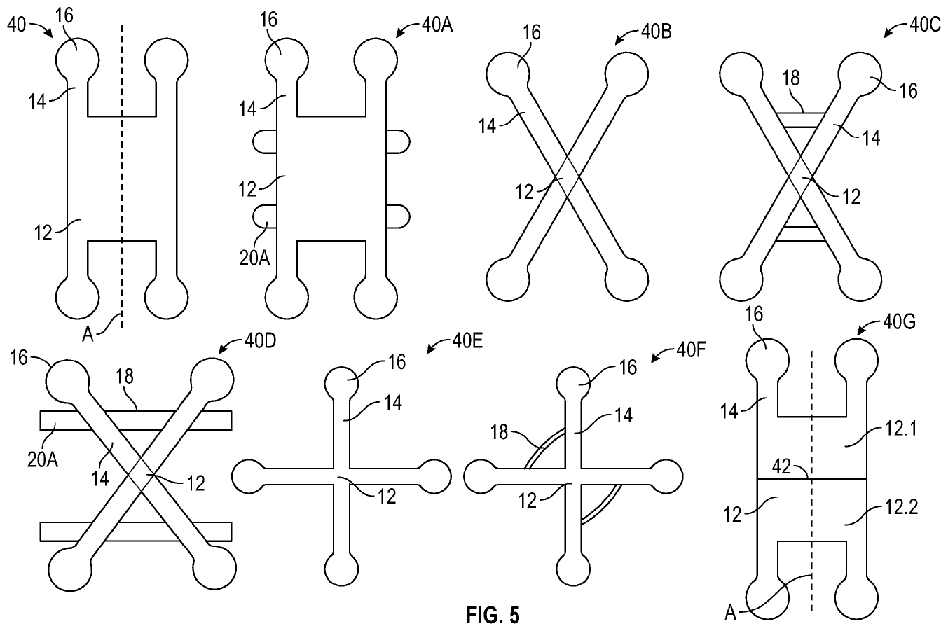

FIG. 5 is a schematic view of various configurations of a four prong nasal swab apparatus according to the invention. The apparatus having four prongs enables two independent uses for the cleaning, washing, prepping and/or sterilizing of the human nose. An apparatus 40 has the body 12 and four of the prong 14 extending along the longitudinal axis A. Pairs of the prongs 14 extend from the body 12 in opposite directions. An apparatus 40A is similar to the apparatus 40 with four of nasal alar guides 20A attached. An apparatus 40B has an X-shape with the angles between the prongs 14 in each pair of the prongs being less than 90°. An apparatus 40C is similar to the apparatus 40B with the nasal columellar guide 18 attached to each pair of the prongs 14. An apparatus 40D is similar to the apparatus 40C with four of the guides 20A added. An apparatus 40E is similar to the apparatus 40B with the prongs 14 spaced apart by 90°. An apparatus 40F is similar to the apparatus 40E with the nasal columellar guide 18 attached to each pair of the prongs 14. An apparatus 40G is similar to the apparatus 40, but has a tear or fold line 42 that extends transverse to the longitudinal axis A. When the line 42 is formed as a tear line, the body 12 can be separated into an upper body portion 12.1 and a lower body portion 12.2. The separate body portions 12.1, 12.2 function like two of the dual prong apparatus 10 shown in FIG. 1. When the line 42 is formed as a fold line, the body 12 can be folded such that the upper body portion 12.1 and the lower body portion 12.2 are adjacent. This configuration reduces the profile to achieve a compact package for shipping and storage. Also, when one of the body portions 12.1, 12.2 is being used, the fold line 42 permits the other body portion to be rotated away from the user's face to prevent undesired contact. Other configurations of the four prong nasal swab apparatus are possible.

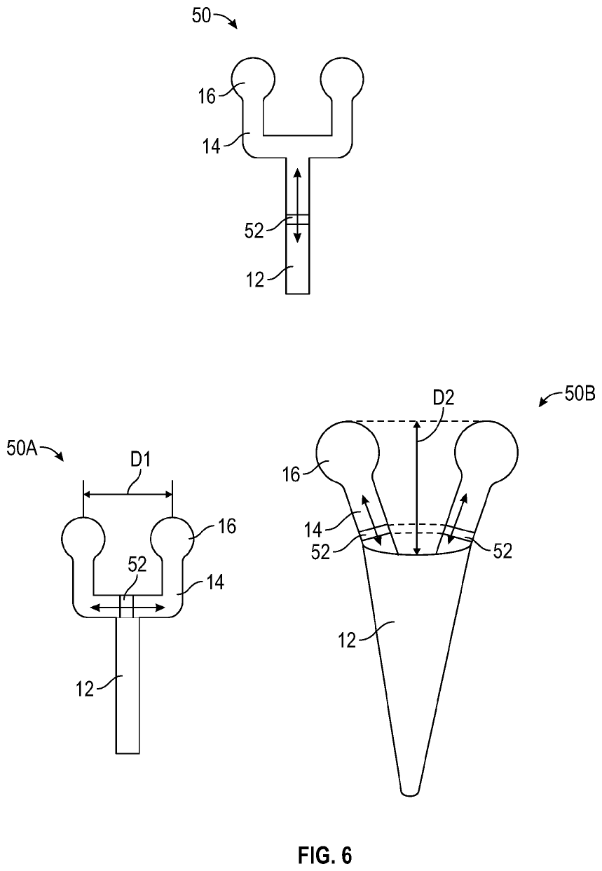

FIG. 6 is a schematic view of various configurations of a nasal swab apparatus according to the invention that provides adjustability of the prongs, the body and the nasal guides. The adjustability is provided by any suitable means such as parts that telescope, slide, click, ratchet, etc. An adjustable nasal swab apparatus 50 includes an adjustment mechanism 52 positioned in the body 12 to enable parts of the body to move relative to one another in the directions of the double arrow to change a length of the body. An adjustable nasal swab apparatus 50A includes the adjustment mechanism 52 positioned in horizontal portions of the prongs 14 to enable parts of the prongs to move relative to one another to change the prong spacing D1. An adjustable nasal swab apparatus 50B includes the adjustment mechanism 52 positioned in the prongs 14 to enable parts of the prongs to move relative to one another to change the insertion depth D2. Other configurations of the adjustment mechanism are possible.

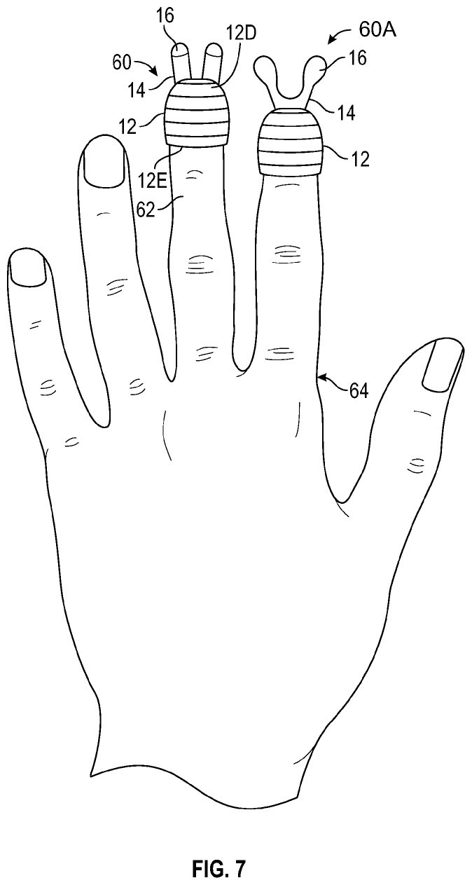

FIG. 7 is a schematic view of various configurations of a thimble style nasal swab apparatus according to the invention. A nasal swab apparatus 60 has a body 12 formed as a finger cap shaped like a thimble. A pair of the prongs 14 with the swabs 16 are attached at a closed end 12D of the body 12. An open end 12E of the body 12 receives the end of a finger 62 on a human hand 54. The body 12 can be made of an elastic material to secure the apparatus 60 on the finger 62 during use. A nasal swab apparatus 60A is similar to the apparatus 60, but the two prongs 14 are formed as an integral unit. In a variation, the swab is incorporated in a finger sleeve.

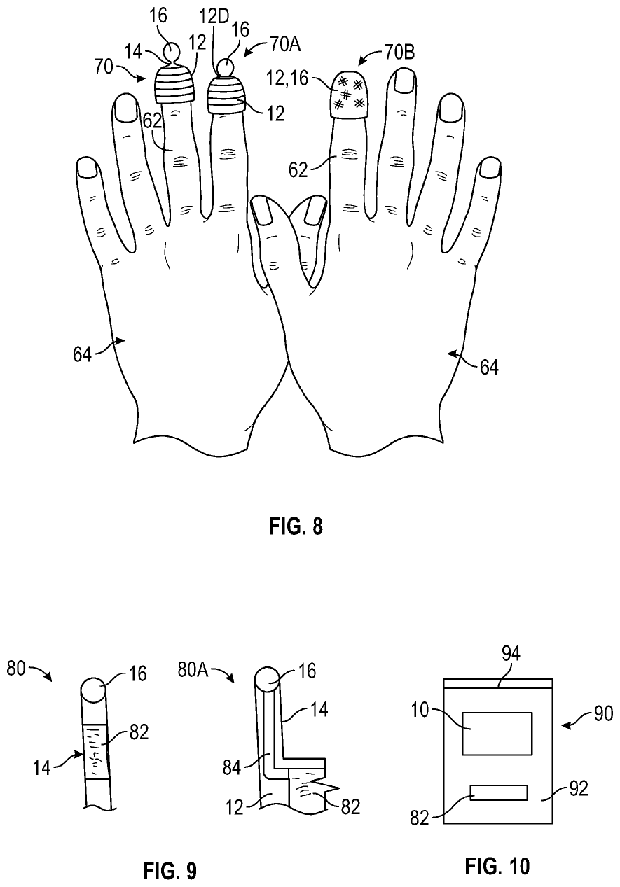

FIG. 8 is a schematic view of configurations of a single prong thimble style nasal swab apparatus according to the invention. A nasal swab apparatus 70 is similar to the apparatus 60 of FIG. 7 but has the single prong 14 with the swab 16. A nasal swab apparatus 70A is similar to the apparatus 70 with the prong 14 integrated into a closed end 12D of the body 12. The swab 16 is directly attached to the closed end 12D of the body 12. Another nasal swab apparatus 70B incorporates the prong 14 and the swab 16 into the body 12. The body 12 of the apparatus 70B is made of a material that carries the antiseptic or disinfectant solution. All of the thimble style nasal swab apparatuses provide the option of providing additional antiseptic or disinfectant solution inside the body 12 for application to the finger 62.

The disinfectant solution can be applied to the swab 16 from a container at the time of use of the nasal swab apparatus. As an alternative, the disinfectant solution can be applied to the swab 16 at the time of packaging the nasal swab apparatus in a leak proof package. Another alternative is shown in FIG. 9 wherein a nasal swab apparatus 80 incorporates a frangible ampule 82 positioned in the prong 14. The frangible ampule 82 contains a quantity of an antiseptic or disinfectant solution 84. Upon breaking the frangible ampule 82, the solution 84 is delivered to the swab 16. Another embodiment nasal swab apparatus 80A has the frangible ampule 82 positioned in the body 12 and connected to the swab 16 by a conduit 84 extending through the prong 14 to deliver the solution 84 to the swab 16.

FIG. 10 shows a nasal swab kit 90 that includes a sealed container 92 enclosing at least one of the nasal swab apparatuses described above. For example, the container 92 can be a pouch enclosing at least one of the nasal swab apparatus 10. The pouch 92 has an opening mechanism 94 for accessing the at least one nasal swab apparatus 10. The opening mechanism 94 can be a tear line extending adjacent to an edge of the pouch 92. The kit 90 also can include one or more of the containers of the antiseptic or the disinfectant such as the frangible ampule 82 in the pouch 92.

In accordance with the provisions of the patent statutes, the invention has been described in what is considered to represent its preferred embodiment. However, it should be noted that the invention can be practiced otherwise than as specifically illustrated and described without departing from its spirit or scope.

1. A nasal swab apparatus for cleaning, washing, decolonizing, prepping and/or sterilizing of human nose nasal vestibules, the nasal swab apparatus comprising:
a body extending along a longitudinal axis;
at least one prong extending parallel to or along the longitudinal axis of the body, the at least one prong having an attachment end connected to the body and an opposite free end;
a swab attached to the free end of the at least one prong;
a nasal guide being a nasal columellar guide or a nasal alar guide, the nasal guide being connected to the body or to the at least one prong; and
wherein the body is configured to be engaged by a human hand to position the swab into a selected one of the nasal vestibules of a human nose such that the nasal guide contacts an exterior region of the nose to limit an insertion depth of the swab.

2. The nasal swab apparatus according to claim 1 wherein the body includes a receptacle and the attachment end of the at least one prong is fixed in the receptacle or is removably retained in the receptacle.

3. The nasal swab apparatus according to claim 1 wherein the at least one prong is a first prong extending parallel to the longitudinal axis of the body, including a second prong extending parallel to the longitudinal axis of the body, the second prong having an attachment end connected to the body and a free end, the second prong being spaced a predetermined distance from the first prong, and including another swab attached to the free end of the second prong.

4. The nasal swab apparatus according to claim 3 wherein nasal guide is the nasal columellar guide that either is formed as a portion of an attachment edge of the body between where the first and second prongs are connected to the body or extends between the first and second prongs and is spaced from the body.

5. The nasal swab apparatus according to claim 3 wherein the nasal guide is the nasal alar guide that either extends from the body or extends from one of the first and second prongs.

6. The nasal swab apparatus according to claim 1 wherein the nasal guide is the nasal alar guide that extends from the at least one prong.

7. The nasal swab apparatus according to claim 1 wherein the body and/or the at least one prong are constructed from a material selected from a paper, a paper product, a plastic, a metal and a synthetic product.

8. The nasal swab apparatus according to claim 7 wherein the material is biodegradable.

9. The nasal swab apparatus according to claim 1 wherein the swab is adapted to hold a quantity of an antiseptic or a disinfectant and to release the antiseptic or the disinfectant upon contact with a surface in the nasal vestibule of the human nose.

10. The nasal swab apparatus according to claim 9 including a frangible ampule containing the antiseptic or the disinfectant, the frangible ampule being positioned in the at least one prong or in the body, wherein when the frangible ampule is broken, the antiseptic or the disinfectant is delivered to the swab.

11. The nasal swab apparatus according to claim 1 including an adjustment mechanism positioned in the body and enabling selective adjustment of the body in a direction of the longitudinal axis.

12. The nasal swab apparatus according to claim 1 including an adjustment mechanism positioned in the at least one prong and enabling selective adjustment of the at least one prong in a direction of the longitudinal axis of the body or in a direction transverse to the longitudinal axis of the body.

13. The nasal swab apparatus according to claim 1 wherein the swab is made of an absorbent material including any of cotton or other natural product, foam, sponge, synthetic fiber and gauze.

14. The nasal swab apparatus according to claim 1 wherein the at least one prong is one of four prongs connected to the body, a first pair of the prongs extending in one direction parallel to the longitudinal axis of the body and another pair of the prongs extending in an opposite direction.

15. The nasal swab apparatus according to claim 1 wherein the at least one prong is one of four prongs connected to the body, the prongs extending from the body to form an X-shape or the prongs extending from the body at a 90° spacing.

16. The nasal swab apparatus according to claim 1 wherein the body is formed as a finger cap adapted to be removably retained on an end of a human finger.

17. The nasal swab apparatus according to claim 16 wherein the body has a closed end and either the prong is formed integral with the closed end or the prong and the swab are incorporated into the body.

18. A nasal swab kit comprising:
at least one of the nasal swab apparatus according to claim 1; and
a sealed package enclosing the at least one nasal swab apparatus, the package having an opening mechanism for accessing the at least one nasal swab apparatus.

19. The nasal swab kit according to claim 18 wherein the package is a pouch and the opening mechanism is a tear line extending adjacent to an edge of the pouch.

20. The nasal swab kit according to claim 18 including a container of the antiseptic or the disinfectant enclosed in the package.
